(19) United States
(12) Patent Application Publication
(10) Pub. No.: US 2006/0246116 A1
(43) Pub. Date: Nov. 2, 2006

(54) Formula and method for the delivery of medications to animals

(75) Inventor: Lawrence J. Putz, Grand Junction, CO (US)

Correspondence Address: MARTIN & HENSON, P.C., 9250 W 5TH AVENUE, SUITE 200, LAKEWOOD, CO, 80226, US

(73) Assignee: Petworks, LLC, Grand Junction, CO (US)

(21) Appl. No.: 10/565,095

(22) PCT Filed: Jul. 14, 2004
(86) PCT No.: PCT/US04/22851

§ 371 Date: May 30, 2006

(30) Foreign Application Priority Data
Jul. 18, 2003 (US) 60488592

(51) Int. Cl. A61K 47/00

(52) U.S. Cl. 424/442

(57) ABSTRACT

A compound is used to conceal a medication or supplement to be given to an animal. The compound includes water as a carrier solvent, at least one hydrogenated oil, at least one emulsifier and a preservative. One or more additional ingredients may also be optionally added to the compound. These ingredients include anti-caking agents, viscosifiers, flavorings, coloring agents and binders. In any event, the embodiments may have a peel strength to paper of about 0.079 lbs./inch or a peel strength to itself of about 0.079 lbs./inch. It may have a tensile strength of about 0.33 psi and a compressive strength of about 2.02 psi. The invention also includes a method of administering a medication/supplement to an animal by forming a layer of the compound around the medication/supplement.

### FIELD OF THE INVENTION

The present invention broadly concerns the administration of medications and supplements to animals, including humans. The invention particularly is directed to a compound formulated for use as a carrier of medication/supplement and a method of utilizing such a compound. This invention specifically is directed to a compound and a method of use of that compound whereby the medication/supplement, in the form of a pill, capsule, liquid and the like, may be administered to an animal, such as a dog or cat. The invention also concerns a product in the form of a medication/supplement coated with the compound.

### BACKGROUND OF THE INVENTION

Since the domestication of animals in pre-historic times, humans have enjoyed many benefits provided by domesticated animals. While many animals are raised as food sources, other animals are desired as pets or companions. Primary among companion animals are both dogs and cats. In early Egyptian times, for example, dogs were used to assist in hunting as well as being emotional companions. Cats, on the other hand, were not only kept as pets, but in some senses were deified. In more modem times, cats and dogs are found in a substantial number of homes in virtually every country throughout the world.

Regardless of the reason for human association with animals, it is not unusual for the animal to become ill, from time to time. In such circumstances, it is common to give the animals medication to treat the illness, to relieve pain and like. Even when the animal is healthy, it is not uncommon for a person to give the animal a nutritional supplement such as a vitamin, mineral, etc. Indeed, while many of the products of the supplement industry are targeted for human consumption, there is a significant market for supplements for animals. In any event, certain problems arise, however, when attempting to give an animal medication or supplement by the oral route. It is known, for example, that different animals react differently to oral medications and supplements. Even among a specific species, one animal may be more finicky than another animal when it comes to swallowing an oral medication or supplement.

To illustrate this situation, one may again compare cats and dogs. On one hand, cats are known to approach food cautiously and may smell the food for a significant time before actually tasting it. When a cat does decide to taste a food, particularly an unfamiliar food, the cat will nibble and chew at the food, and sometimes spit the food out after it has sampled it. Dogs, on the other hand, are known to have a “gulp” reflex. When a dog takes a food into its mouth, after smelling it, the dog will gulp the food whole provided that the food has a pleasing taste. However, if the taste of the food is not pleasing, a dog will often spit the food out and refuse to swallow it. For this reason, it is difficult to administer oral medications both to cats and to dogs.

In the past, it is known for people to attempt to conceal a medication/supplement or to mask its flavor by encapsulating the solid medication/supplement in a piece of food, such as bread, cheese or hamburger. While this technique is sometimes successful, many times the foodstuff and the medication/supplement separates in the dog's mouth so that the dog detects the presence of the medication/supplement either through taste or tactile sensation. This can cause the dog to disgorge the solid medication/supplement without swallowing, despite the gulp reflex. Other carriers, such as peanut butter, are either too sticky or lack the viscosity to make them ideal carriers for concealing medication.

Accordingly, the present invention addresses the need for an improved compound which may be used to encapsulate a solid or liquid form medication or supplement as a delivery vehicle to an animal. The present invention is directed to a compound for and a method of administering oral medications and supplements to dogs and cats, in particular, but it should be understood that the compounds and methods taught herein may be used to administer medications or supplements to other animals and even humans. It is especially formulated for use with a medication or a supplement in a solid form such as pills, tablets, capsules, caplets and the like, but it can also be used to encapsulate liquid medications and supplements. The invention includes a formulation for such as compound as well as a method employing this compound.

### SUMMARY OF THE INVENTION

It is an object of the present invention to provide a new and useful compound for a method of delivering medication to an animal.

Another object of the present invention is to provide a compound and method for delivering solid form medications to a dog or cat.

A further object of the present invention is to provide a compound of suitable viscosity and tackiness so that a mass of the compound may be molded around a solid form medication yet which compound is not so sticky as to cause the dog to chew the compound.

Still a further object of the present invention is to provide a tasty compound that may be molded around a solid form medication such that, when given to the dog, it triggers the dogs gulp reflex.

Yet another object of the present invention is to provide a medication/supplement product have a coating thereon that facilitates administration of the medication/supplement to an animal.

According to the present invention, then, a compound is provided that is adapted to conceal a medication/supplement when administered to an animal. Broadly, the compound includes water as a carrier solvent, at least one hydrogenated oil, at least one emulsifier and a preservative.

More particularly, if desired, the compound may include an anti-caking agent, and an exemplary anti-caking agent is silicone dioxide. The compound may also include a viscosifier; here, an exemplary viscosifier is sodium carboxymethylcellulose. If desired, the compound may further include a sweetening agent.

In the exemplary embodiments, the hydrogenated oil is selected from a group consisting of vegetable oil and fish oil. The emulsifier is selected from a group consisting of casein, disodium phosphate, mono-glycerides and di-glycerides. The preservative may be any preservative known in the art, but, for example, may be salt and/or sorbic acid.

The compound may also include a flavoring. Here, for example, the flavoring is an artificial flavoring selected from a group consisting of beef, chicken, shrimp, apple, cherry and grape. The compound may also, if desired, include a coloring agent and may include a binder. An example of such a binder is cornstarch.

In the compounds described above, the water may be selected to comprise about forty percent (40%) to sixty percent (60%) by weight. The hydrogenated oil may comprise between about nineteen percent (19%) to twenty-five percent (25%) by weight. The emulsifier may comprise between about fourteen percent (14%) to twenty percent (20%) by weight of the compound.

This compound has physical properties including, for example, a peel strength to paper of about 0.033 pounds per inch. It may also have a peel strength to itself of about 0.079 pounds per inch. The compound may have a tensile strength about 0.33 psi. Further the compound may have a compressive strength of about 2.02 psi.

As noted, the present invention contemplates a product in the form of a dosage of a compound selected from a group consisting of medications and food supplements and a coating for the dosage. This coating compound is as described above.

Further, the present invention is directed to a method of administering a medication/supplement to an animal. Broadly, the method includes a step of providing a mass of selected size of a compound wherein the compound includes water as a carrier solvent, at least one hydrogenated oil, at least one emulsifier and a preservative. The method then includes the step of encapsulating the medication/supplement in the mass whereby the mass forms a layer around the medication/supplement.

In its more detailed description, the method may include the step of flattening the mass into a sheet having a thickness of about 1/16th inch (3.2 mm) to ¼ inch (6.4 mm). The step of encapsulating the medication/supplement is then accomplished by completely wrapping the medication/supplement in the sheet. If desired, the sheet may be disk shaped. Alternatively, the step of forming the mass may be accomplished by forming the mass into a generally spherical shape. The step of encapsulating the medication/supplement is then accomplished by creating a cavity in the mass, placing the medication/supplement in the cavity and, thereafter, sealing the cavity to form the layer. In any event, the medication/supplement may be in tablet form, and the method may include the step of crushing the tablet form to granulate the medication/supplement prior to encapsulating the medication/supplement.

These and other objects of the present invention will become more readily appreciated and understood from a consideration of the following detailed description of the exemplary embodiments of the present invention, in which:

### DETAILED DESCRIPTION OF THE EXEMPLARY EMBODIMENTS

The present invention broadly relates to the provision of medications and supplements to animals, including humans. It should be specifically understood that the term “animal” thus includes humans as well as other animals, both domesticated and wild. Moreover, as used herein, the term “mediation/supplement” will mean those compounds administered to treat injury, illness, pain, etc., as well as those compounds thought to promote good health, such as vitamins, minerals and other food supplements. The compound may also be employed with liquid medications and supplements without departing from the scope of this invention.

More specifically, however, the present invention concerns a formulated compound and a method for encapsulating tabletized medications/supplements, and the like, such as those commonly given to pets. The invention especially useful as a composition and a new use for the composition as a carrier for pills or other tablets to be given to a dog or a cat. The invention thus contemplates a product in the form of a medication/supplement that is coated with the compound.

The compound according to the formulations of the present invention can be prepared either in a heated procedure or at room temperature, but certain of the ingredients must be altered between the two formulation methods. Generally, the formulation requires, at a minimum, the following ingredients: (1) water; (2) hydrogenated oil; (3) at least one emulsifier; and (4) a preservative. Usually it is desirable to add a flavoring, as well.

The formulations according to the present invention can be made either by a hot mix technique or by a cold mix technique. Generally, the hot mix formulations use from about 40% to 60% water by weight, from about 20% to 25% by weight of hydrogenated oil, from about 16% to 20% by weight of emulsifier and about 0.2% to 0.3% by weight of the preservative. The cold melt formulation has about 70% by weight water, about 13.5% by weight of a leavening agent, such as brewers yeast and about 12% of a gelling agent like Sodium CMC or other suitable one and a preservative like Sorbic acid at about 0.02%.

### A. Hot Mix Formulations

The present invention is best formulated in a heated environment of between about 140° to 190° Fahrenheit (about 60° to 88° Celsius). This hot formulation is thought to need water, hydrogenated oils, emulsifiers and a preservative. With greater detail, the hot formulation contains water, hydrogenated vegetable oil, hydrogenated fish oil, casein, mono and diglycerides, disodium phosphate, sorbic acid and a flavoring. Sodium carboxymethylcellulose is useful in adjusting the relative tackiness and stickiness of the final compound. However, excess sodium carboxymethylcellulose can act as a laxative when administered to an animal, such as a pet dog, so that care must be used in the formulation. In addition, sodium carboxymethylcellulose can cause some degree of flatulence in the animal. Thus, it can be desirable to substitute silicon dioxide for the carboxymethylcellulose.

1. Hot Mix Formulation I

With still greater detail the following list sets forth the formulation along with an identification of the purpose of each ingredient in a first exemplary hot mix formulation:

| Ingredient | Amount | Purpose |
|---|---|---|
| Water | 228 lbs. | Carrier/Solvent |
| Hydrogenated | 95 lbs. | Emulsifier |
| Vegetable Oil | | |
| Casein | 68 lbs. | Emulsifier |
| Sorbitol | 34 lbs. | Sweet flavor and |
| | | Humectant |
| Mannitol | 19 lbs. | Sweet flavor and |
| | | Anti-caking agent |
| Corn Starch | 17 lbs. | Binder |
| Disodium Phosphate | 15 lbs. | Emulsifier |
| Sugar | 14 lbs. | Sweetener |
| Hydrogenated | 14 lbs. | Binder/Emulsifier |
| Fish Oil | | |
| Mono and diglycerides | 6 lbs. | Emulsifier |
| | | (BFT 74A) |
| Salt | 2 lbs. | Preservative and |
| | | Flavoring |
| Natural flavoring (beef) | 2 lbs. | Palatant |
| Sodium Carboxymethyl- | 2 lbs. | Binder/Viscofier |
| cellulose¹ | | |
| Sorbic Acid² | 1.5 lbs. | Preservative |
| Iron Oxide³ | 1 lb. | Color additive |

¹Levels of sodium carboxymethylcellulose should not exceed 0.50% by weight
²Levels of sorbic acid should be in a range of about 0.20% to 0.30% by weight
³Levels of iron oxide should not exceed about 0.25% by weight

To prepare this formulation, all but about four gallons of the water is heated in a jacketed paddle mixer to the desired temperature of between about 140° to 150° Fahrenheit (60° to 65° Celsius). The sorbitol, sugar and cornstarch are then mixed in a paddle mixer under agitation and this mixture is slowly heated to about 185° Fahrenheit (85° Celsius) over a period of about one hour. During this time, a preparation is prepared by placing the disodium phosphate in the four gallons of water that had been set aside.

At the end of the one-hour period, the mono and diglyceride emulsifier, such as BFT 74A (formerly Atmul 84) obtained from American Ingredients Company of Kansas City, Mo., and the composition is stirred under heat for about two minutes. After this, the casein and the mannitol are added, and the mixture is stirred under heat for another five-minute period. When this time has elapsed, the preparation of four gallons water and the disodium phosphate is mixed in. This combination is stirred for fifteen minutes while the temperature is returned to about 185° Fahrenheit (85° Celsius).

At the end of the fifteen-minute period, the hydrogenated vegetable oil and the hydrogenated fish oil (wax) are added into the mixture, and the resulting mixture is stirred for another fifteen minutes. At the end of this time, the sorbic acid, salt and the remaining additives (beef flavoring and iron oxide) and the sodium carboxymethylcellulose are mixed in to finish the product. The finished product may be placed in an extruder, and the final composition extruded and packaged.

It may be noted that the addition of sodium carboxymethylcellulose is employed to adjust the tackiness of the composition to a desired level. However, due to its laxative effect, care must be used to ensure that the levels do not exceed governmental regulations. In any event, this composition has been found to be highly palatable for dogs, and the product does not normally separate from the medication in a dog's mouth upon administration. It may be desirable, also, to include a palatant that will enhance the aroma to humans, as well. Here, a suitable palatant may be BioFlavor C-20069, obtainable from BioProducts, Inc. of Fairlawn, Ohio. If used, it should in the amount of about two pounds and be added at the same time as the beef flavoring.

Accordingly, the present invention relates to the use of this composition as a delivery vehicle for medications/supplements, especially those that are in unitary dosage form. By this it is meant that the medication/supplement is in the form of a tablet, a capsule, a gel capsule and the like as opposed to liquid form, although the present invention may be used with liquids.

2. Hot Mix Formulation II

While the compound developed as Hot Mix Formulation I is quite useful for its intended purpose when fresh, it was learned that over an extended period of time, the compound may harden because of the natural beef flavoring palatant. Accordingly, a second hot mix formulation was developed to address these issues. Also, the sodium carboxymethylcellulose was omitted and an anti-caking agent in the form of silicon dioxide was substituted. Less water was therefore used. This also removed any issue of the laxative effect of the sodium carboxymethylcellulose or its tendency to cause flatulence. An artificial flavoring was substituted for the previously-used natural beef flavoring. This flavoring can be of any desired taste, but beef, chicken and shrimp flavoring agents have been used where the target animal is a dog or a cat. Where a human child may receive the delivery system as an adjunct to taking a medication or supplement, the desired flavoring agent may be different, such as a fruit flavor (apple, cherry, grape, etc.)

The ingredients for the second hot mix formulation along with an identification of the purpose of each ingredient, then, is:

| Ingredient | Amount | | Purpose |
|---|---|---|---|
| Water¹ | 200 | lbs. | Carrier/Solvent |
| Hydrogenated | 95 | lbs. | Emulsifier |
| Vegetable Oil¹ | | | |
| Casein | 68 | lbs. | Emulsifier |
| Sorbitol | 34 | lbs. | Sweet flavor and |
| | | | Humectant |
| Mannitol | 19 | lbs. | Sweet flavor and |
| | | | Anti-caking agent |
| Corn Starch | 17 | lbs. | Binder |
| Disodium Phosphate | 15 | lbs. | Emulsifier/ |
| | | | Suspending Agent |
| Sugar | 14 | lbs. | Flavoring |
| | | | (Sweetener) |
| Hydrogenated | 14 | lbs. | Binder/Emulsifier |
| Fish Oil | | | |
| Mono and diglycerides¹ | 6 | lbs. | Emulsifier |
| | | | (BFT 74A) |
| Salt | 2 to 4 | lbs. | Preservative and |
| | | | Flavoring |
| Aritificial flavoring | 2 | lbs. | Palatant |
| (liquid) | | | |
| Silicon dioxide² | 2 | lbs. (max) | Anticaking agent |
| Sorbic Acid³ | 1.5 | lbs. | Preservative |
| Iron Oxide⁴ | 1 | lb. | Color additive |

¹Water comprises between about 40% to 60% by weight of the compound, the hydrogenated oil comprises between about 19% to 25% by weight of the compound, and the emulsifier comprises between about 14% to 20% by weight of the compound.
²Levels of silicon dioxide should not exceed 1.0% by weight
³Levels of sorbic acid should be in a range of about 0.20% to 0.30% by weight
⁴Levels of iron oxide should not exceed about 0.25% by weight

To prepare this formulation, all but about four gallons of the water is heated in a jacketed paddle mixer to the desired temperature of between about 140° to 150° Fahrenheit (60° to 65° Celsius). The sorbitol, sugar and corn starch are then mixed in a paddle mixer under agitation and this mixture is slowly heated to about 185° Fahrenheit (85° Celsius) over a period of about one hour. During this time, a preparation is prepared by placing the disodium phosphate in the four gallons of water that had been set aside.

At the end of the one hour period, the mono and diglyceride emulsifier, such as BFT 74A (formerly Atmul 84) obtained from American Ingredients Company of Kansas City, Mo., and the composition is stirred under heat for about two minutes. After this, the casein and the mannitol are added, and the mixture is stirred under heat for another five minute period. When this time has elapsed, the preparation of four gallons water and the disodium phosphate is mixed in. This combination is stirred for fifteen minutes while the temperature is returned to about 185° Fahrenheit (85° Celsius).

At the end of the fifteen minute period, the hydrogenated vegetable oil and the hydrogenated fish oil (wax) are added into the mixture, and the resulting mixture is stirred for another fifteen minutes. At the end of this time, the sorbic acid, salt and the remaining additives (flavoring and iron oxide) and the silicon dioxide are mixed in to finish the product. The finished product may be placed in an extruder, and the final composition extruded and packaged. Again, it may be desirable to include a palatant that will enhance the aroma to humans, as well. If used, it should in the amount of about two pounds and be added at the same time as the primary flavoring.

3. Properties of Hot Mix Formulation II

A test was conducted on a sample of the Hot Mix Formulation II to determine some of its physical characteristics of it as a coating. These physical characteristics included peel strength (both to itself and to paper), tensile strength, compressive strength and penetration.

Peel strength was tested using a modified ASTM D903-98. A sample of the compound was pressed into a sheet having a 0.1 inch nominal thickness. One inch wide strips were then cut for use as specimens. To test peel strength to itself, two strips were pressed together in one pass using a 4½ pound roller with a cross-head speed of twelve inches per minute. To test peel strength to paper, one strip was pressed onto a one inch wide piece of paper (Mil-B-131H, Class 2, Marvel Seal 1312, Lot No. H-509) in one pass using a 4½ pound roller with a cross-head speed of twelve inches per minute.

Tensile strength was tested according to modified ASTM D882-02. Again, a sample was pressed into a sheet having a 0.1 inch nominal thickness. One inch wide strips were then cut for use as specimens. The testing was conducted using a cross-head speed of two inches per minute and an initial grip separation of one inch.

Compression was tested according to modified ASTM D695-02a. Cylindrical specimens were formed with a diameter of 0.75 inches at a height of 0.6 inches. The specimens were compressed at a rate of 0.05 inches per minute.

Finally, cone penetration was tested according to modified ASTM D217-02. The sample was pressed into a cylindrical tin with a diameter of 1.95 inches and a height of 1.29 inches. A twenty degree cone was used with a 47.5 gram spindle for all penetration testing.

The results of these tests are summarized in the following Table I:

TABLE I

| Test | Result | |
|---|---|---|
| Peel Strength to self | 0.079 | lbs./inch |
| Peel Strength to paper | 0.033 | lbs./inch |
| Tensile Strength | 0.33 | psi |
| Compressive Strength | 2.02 | psi |
| Penetration | 15.5 | mm |

### B. Cold Mix Formulation

A less desirable formulation was obtained using a cold mix method. Here, the following list sets forth the formulation along with an identification of the purpose of each ingredient:

| Ingredient | Amount | | Purpose |
|---|---|---|---|
| Water | 58.5 | lbs | Carrier/Solvent |
| Brewer's yeast | 11.25 | lbs | Leavening Agent |
| Sodium Carboxymethyl- | 10 | lbs | Binder/viscosifier |
| cellulose | | | |
| Sodium benzoate | 14 | oz | Preservative |
| Sorbic acid | 14 | oz | Preservative |
| Artificial flavoring | 1 | lb | Palatant |
| Iron Oxide | 0.5 | lb | Color additive |

In this formulation, the ingredients are added to water at room temperature in a paddle mixer and mixed thoroughly. Again, an aromatic palatant may be added to enhance the smell to humans. A disadvantage of the cold mix formulation is that the amount of sodium carboxymethylcellulose may exceed that recommended for use with animals due to its laxative effect

### C. Methods of Use

The method of the present invention involves the use of the above-described compositions as a delivery vehicle for oral medication/supplement to animals, such as a pet dog. Broadly, the method includes selecting a desired quantity of the composition, as described above, of a sufficient amount to form a layer of composition that can completely encase the unit dosage of medication/supplement. Of course, this quantity depends on the volumetric size of the unit dosage. The selected quantity is then formed about the unit dosage by any convenient technique. The composition should have a consistency such that it may be molded into a plug that retains its shape upon molding and wherein the compound has suitable tackiness to adhere to itself but is not so sticky as to adhere to a person's fingers or to a table top or other surface.

The volume of the compound is selected based upon the size of medication/supplement in pill, tablet, capsule, caplet or other unit dosage form to be given to the animal. The volume is selected such that, upon encapsulating the medication/supplement, the compound forms a layer completely encasing the medication/supplement with a layer thickness of between about 1/16^{th} inch to ¼^{th} inch. Thus, the selected mass of compound should have a volume that approximates the surface area of the solid medication multiplied by the desired layer thickness.

For example, after the mass of composition is selected, it may be flattened into a thin disc at a thickness about equal to the desired layer thickness. The medication/supplement is placed on the flattened sheet at a central location, and the margin of the sheet that is located between the medication/supplement and the periphery of the sheet is folded over the medication/supplement so that the peripheral edges are in close proximity to one another. The margin is then molded to place the peripheral edges in contact with one another, and the edges are sealed to each other due to the tackiness of the compound to form a plug. If desired, the plug may be rolled into a more ball-like presentation.

Alternatively, the selected quantity can be rolled into a somewhat spherical mass, and a cavity can then be formed in the mass. The unit dosage is then place in the cavity, and the mouth of the cavity sealed by pinching the sidewalls together. The encased unit dosage of medication/supplement is then presented to the animal for ingestion. Again, the plug may be rolled into a more ball-like presentation.

In either case, the medication/supplement should be entirely encased by the composition of this invention so that the animal will neither taste nor sense the presence of the medication/supplement. This may work well for many animals; however, some animals may still object to the tabletized form of the medication/supplement. In this case, it is possible to crush the tablet before encapsulating it. Care must be taken, though, that the medication/supplement is in a form that may be crushed without adverse effect. For example, time-released medications should not be crushed.

The plug (whether balled or not) is then administered to the animal. Where the animal, such as a dog, has a high gulp reflex, the animal will tend to swallow the plug and medication/supplement in whole without chewing. The tackiness without stickiness of the plug facilitates this response.

From this description of the method, it should be appreciated that the final composition should have the tackiness to adhere to itself sufficiently so that the composition doe not come apart in the animal's mouth yet not be so sticky as to adhere to the human skin when being molded about the unit dosage. Moreover, it is desired that the layer of composition that covers the unit dosage be about 1/16^{th} inch to ¼^{th} inch in thickness.

### D. Combination Product

The present invention further contemplated a product in the form of a medication/supplement that is encapsulated according to one of the methods described above using a coating of a compound as described above. Here, the product comprises a dosage of a compound selected from a group consisting of medications and food supplements, and a coating substantially enclosing said dosage wherein said coating includes: (1) water as a carrier solvent; (2) at least one hydrogenated oil; (3) at least one emulsifier; and (4) a preservative.

The product may further include an anti-caking agent, such as silicon dioxide. The product may include a viscosifier, such as sodium carboxymethylcellulose. The emulsifier may be selected from a group consisting of casein, disodium phosphate, mono-glycerides, and di-glycerides. A binder, such as corn starch, may also be employed, if desired.

### E. Addtional Uses

The formulations of the present invention may also be employed as a rodent bait or as a carrier for a rodent poison. When used as a bait, the moldablility of the compound allows it to be bound to the tripping mechanism, for example, of a rodent or other animal trap. Due to its adherence, the target pest cannot easily dislodge the compound without triggering the trap. Alternatively, a poison can be encapsulated by the compound and/or the compound can be used as a carrier for the poison for delivery and ingestion by the pest.

Accordingly, the present invention has been described with some degree of particularity directed to the exemplary embodiments of the present invention. It should be appreciated, though, that the present invention is defined by the following claims construed in light of the prior art so that modifications or changes may be made to the exemplary embodiments of the present invention without departing from the inventive concepts contained herein.

I claim:

1. A compound adapted to conceal a medication/supplement when administered to an animal, comprising:
(A) water as a carrier solvent;
(B) at least one hydrogenated oil;
(C) at least one emulsifier, and
(D) a preservative.

2. A compound according to claim 1 including an anti-caking agent.

3. A compound according to claim 2 wherein said anti-caking agent is silicon dioxide.

4. A compound according to claim 1 including a viscosifier.

5. A compound according to claim 4 wherein the viscosifier is sodium carboxymethylcellulose.

6. A compound according to claim 1 wherein said hydrogenated oil is selected from a group consisting of vegetable oil and fish oil.

7. A compound according to claim 1 wherein said emulsifier is selected from a group consisting of casein, disodium phosphate, mono-glycerides, and di-glycerides,

8. A compound according to claim 1 wherein said preservative is selected from a group consisting of salt and sorbic acid.

9. A compound according to claim 1 including a flavoring.

10. A compound according to claim 9 wherein said flavoring is an artificial flavoring selected from a group consisting of beef, chicken, shrimp, apple, cherry, and grape.

11. A compound according to claim 1 including a coloring agent.

12. A compound according to claim 1 including at least one binder.

13. A compound according to claim 12 wherein said binder is corn starch.

15. A compound according to claim 1 wherein the water comprises between about 40% to 60% by weight of the compound.

16. A compound according to claim 1 wherein the hydrogenated oil comprises between about 19% to 25% by weight of the compound.

17. A compound according to claim 1 wherein the emulsifier comprises between about 14% to 20% by weight of the compound.

18. A compound according to claim 1 wherein said compound has a peel strength to paper of about 0.033 lbs./inch.

19. A compound according to claim 1 wherein said compound has a peel strength to itself of about 0.079 lbs./inch.

20. A compound according to claim 1 wherein said compound has a tensile strength of about 0.33 psi.

21. A compound according to claim 1 wherein said compound has a compressive strength of about 2.02 psi.

22. A compound adapted to conceal a medication/supplement when administered to an animal, comprising:
(A) water as a carrier solvent;
(B) hydrogenated vegetable oil;
(C) hydrogenated fish oil;
(D) an emulsifier selected from a group consisting of casein, disodium phosphate, mono-glycerides, and di-glycerides;
(E) corn starch;
(F) a sweetening agent;
(G) a flavoring agent; and
(H) a preservative.

23. A compound according to claim 22 including silicon dioxide as an anti-caking agent.

24. A compound according to claim 22 wherein the water comprises between about 40% to 60% by weight of the compound, the hydrogenated oil comprises between about 19% to 25% by weight of the compound, and the emulsifier comprises between about 14% to 20% by weight of the compound.

25. A compound according to claim 22 including sodium carboxymethylcellulose as a viscosifier, said sodium carboxymethylcellulose comprising about no more than 0.5% of the compound by weight.

26. A product adapted for administration to an animal, comprising:
(A) a dosage of a compound selected from a group consisting of medications and food supplements; and
(B) a coating substantially enclosing said dosage wherein said coating includes
(1) water as a carrier solvent;
(2) at least one hydrogenated oil;
(3) at least one emulsifier; and
(4) a preservative.

27. A product according to claim 26 including an anti-caking agent.

28. A product according to claim 27 wherein said anti-caking agent is silicon dioxide.

29. A product according to claim 26 including a viscosifier.

30. A product according to claim 29 wherein the viscosifier is sodium carboxymethylcellulose.

31. A product according to claim 26 wherein said emulsifier is selected from a group consisting of casein, disodium phosphate, mono-glycerides, and di-glycerides.

32. A product according to claim 26 including at least one binder.

33. A product according to claim 32 wherein said binder is corn starch.

34. A method of administering a medication/supplement to an animal, comprising:
(A) providing a mass of a selected size of a compound including:
(1) water as a carrier solvent,
(2) at least one hydrogenated oil;
(3) at least one emulsifier; and
(4) a preservative; and
(B) encapsulating the medication/supplement in said mass whereby said mass forms a layer around the medication/supplement.

35. A method according to claim 34 including the step of flattening said mass into a sheet having a thickness of between about 1/16 inch ( 3.2 mm) to ¼ inch (6.4 mm), said step of encapsulating the medication/supplement being accomplished by completely wrapping the medication/supplement in said sheet.

36. A method according to claim 35 wherein said sheet is disc-shaped in configuration.

37. A method according to claim 34 including the step of forming said mass into a generally spherical shape, said step of encapsulating the medication/supplement being accomplished by creating a cavity in said mass, placing the medication/supplement in the cavity and thereafter sealing the cavity to form the layer.

38. A method according to claim 34 wherein the medication/supplement is in a tablet form and including the step of crushing the tablet form to granulate the medication/supplement prior to encapsulating the medication/supplement.
